(19) United States
(12) Patent Application Publication
(10) Pub. No.: US 2025/0125049 A1
(43) Pub. Date: Apr. 17, 2025

(54) METHOD AND APPARATUS FOR ASSISTING IN AUTISM SPECTRUM DISORDER DIAGNOSIS BASED ON ARTIFICIAL INTELLIGENCE

(71) Applicant: ELECTRONICS AND TELECOMMUNICATIONS RESEARCH INSTITUTE, Daejeon (KR)

(72) Inventors: Cheol-Hwan YOO, Daejeon (KR); Moon-Ki BACK, Daejeon (KR); Jang-Hee YOO, Daejeon (KR); Byung-Ok HAN, Daejeon (KR)

(21) Appl. No.: 18/795,543

(22) Filed: Aug. 6, 2024

(30) Foreign Application Priority Data
Oct. 17, 2023 (KR) 10-2023-0138218

(51) Int. Cl. G16H 50/20; G16H 30/40

(52) U.S. Cl. CPC G16H 50/20; G16H 30/40

(57) ABSTRACT

Disclosed herein is a method for assisting in Autism Spectrum Disorder (ASD) diagnosis. The method includes transmitting social-interaction-inducing content, receiving input images containing a response of an assessment subject to the social-interaction-inducing content, receiving an ASD diagnosis result for a preset number of input images, among the received input images, and outputting a diagnostic assistive result for the received input images using ASD diagnosis input for the preset number of input images and a pretrained global ASD diagnosis model.

### CROSS REFERENCE TO RELATED APPLICATION

This application claims the benefit of Korean Patent Application No. 10-2023-0138218, filed Oct. 17, 2023, which is hereby incorporated by reference in its entirety into this application.

### BACKGROUND OF THE INVENTION

### 1. Technical Field

The present disclosure relates to a personalization-technology-based telemedicine diagnosis method and apparatus that uses social-interaction-inducing content for early diagnosis of Autism Spectrum Disorder (ASD).

More particularly, the present disclosure relates to a method for assisting a medical expert in diagnosis based on a learning model for ASD diagnosis and providing a personalized model suitable for the inclination of an assessment subject and the medial expert using a small number of diagnostic values.

### 2. Description of Related Art

The Diagnostic and Statistical Manual of Mental Disorders (DSM) published by the American Psychiatric Association defines the characteristics of children with Autism Spectrum Disorder (ASD) as two main characteristics, which are restricted and repetitive behaviors and persistent deficits in social communication and social interaction in a variety of contexts. In particular, lack of nonverbal communication behaviors used for social interaction (eye contact, name call response, social smiles, pointing gestures, and the like) acts as an important diagnostic criterion to diagnose children with ASD.

The prevalence of children with ASD continues to increase every year around the world, and as with other diseases, early diagnosis of children with ASD is very important in terms of providing an opportunity for the brain of a child to change into a normal form during a period of high plasticity and preventing the accumulation of behavioral problems capable of causing discomfort and problems in social interaction with others in advance. However, the current ASD diagnosis systems rely solely on continuous observation of a child by a medical expert, interviews with parents, etc., and in such an environment, the reluctance of children and parents to visit hospitals and undergo diagnosis, long waiting for appointments, and the like lead to a problem of missing the golden time for early diagnosis, which is very important for future prognosis.

Also, in the field of medical image analysis using diagnostic assistive AI, which has recently been increasingly used, pretrained AI models fail to reflect the patterns of individual patients and the personal inclination and unique experiences of a medical expert who performs the final diagnosis, so the susceptibility of the medical expert to a result of assistive diagnosis by AI is reduced, which results in a problem of reducing the practical use of AI in the actual clinical setting.

Accordingly, the present disclosure intends to present a personalization-based telemedicine diagnosis method and apparatus for early diagnosis of ASD in order to solve the above-mentioned conventional problems.

### DOCUMENTS OF RELATED ART

(Patent Document 1) Korean Patent Application Publication No. 10-2023-0030810, titled “Data generation method and learning method using the same”.

### SUMMARY OF THE INVENTION

An object of the present disclosure is to provide supportive results for assisting a medical expert in Autism Spectrum Disorder (ASD) diagnosis based on an Artificial Intelligence (AI) neural network.

Another object of the present disclosure is to provide supportive results for assisting in ASD diagnosis that reflect the individual characteristics of an assessment subject and a medical expert.

In order to accomplish the above objects, a method for assisting in Autism Spectrum Disorder (ASD) diagnosis according to an embodiment of the present disclosure includes transmitting social-interaction-inducing content, receiving input images containing a response of an assessment subject to the social-interaction-inducing content, receiving an ASD diagnosis result for a preset number of input images, among the received input images, and outputting a diagnostic assistive result for the received input images using ASD diagnosis result for the preset number of input images and a pretrained global ASD diagnosis model.

Here, outputting the diagnostic assistive result for the received input images may include generating a personalized ASD diagnosis mapping model using the pretrained global ASD diagnosis model and the ASD diagnosis result and outputting the diagnostic assistive result using the personalized ASD diagnosis mapping model.

Here, in the method, the global ASD diagnosis model may be trained using the output diagnostic assistive result.

Here, the method may further include receiving a result of acceptance of the output diagnostic assistive result.

Here, the method may further include transmitting the diagnostic assistive result to the assessment subject.

Here, the ASD diagnosis result may include diagnostic results for eye contact, name call response, a social smile, and pointing.

Here, the global ASD diagnosis model may include respective diagnostic models corresponding to the eye contact, the name call response, the social smile, and the pointing.

Here, outputting the diagnostic assistive result for the received input images may comprise generating inference values for the eye contact, the name call response, the social smile, and the pointing based on the respective diagnostic models corresponding to the eye contact, the name call response, the social smile, and the pointing and outputting a personalized diagnostic assistive result using the personalized ASD diagnosis mapping model.

Also, in order to accomplish the above objects, an apparatus for assisting in ASD diagnosis according to an embodiment of the present disclosure includes one or more processors and executable memory for storing at least one program executed by the one or more processors. The at least one program includes instructions for performing steps of transmitting social-interaction-inducing content, receiving input images containing a response of an assessment subject to the social-interaction-inducing content, receiving an ASD diagnosis result for a preset number of input images, among the received input images, and outputting a diagnostic assistive result for the received input images using ASD diagnosis result for the preset number of input images and a pretrained global ASD diagnosis model.

Here, outputting the diagnostic assistive result for the received input images may include generating a personalized ASD diagnosis mapping model using the pretrained global ASD diagnosis model and the ASD diagnosis result and outputting the diagnostic assistive result using the personalized ASD diagnosis mapping model.

Here, the program may train the global ASD diagnosis model using the output diagnostic assistive result.

Here, the program may further include an instruction for performing a step of receiving a result of acceptance of the output diagnostic assistive result.

Here, the program may further include an instruction for performing a step of transmitting the diagnostic assistive result to the assessment subject.

Here, the ASD diagnosis result may include diagnostic results for eye contact, name call response, a social smile, and pointing.

Here, the global ASD diagnosis model may include respective diagnostic models corresponding to the eye contact, the name call response, the social smile, and the pointing.

Here, outputting the diagnostic assistive result for the received input images may comprise generating inference values for the eye contact, the name call response, the social smile, and the pointing based on the respective diagnostic models corresponding to the eye contact, the name call response, the social smile, and the pointing and outputting a personalized diagnostic assistive result using the personalized ASD diagnosis mapping model.

### BRIEF DESCRIPTION OF THE DRAWINGS

The above and other objects, features, and advantages of the present disclosure will be more clearly understood from the following detailed description taken in conjunction with the accompanying drawings, in which:

FIG. 1 is a flowchart illustrating a method for assisting in Autism Spectrum Disorder (ASD) diagnosis according to an embodiment of the present disclosure;

FIG. 2 is a detailed structural diagram of a personalization-based telemedicine diagnosis system for early diagnosis of ASD;

FIG. 3 conceptually illustrates components of social-interaction-inducing content;

FIG. 4 illustrates a personalization-based telemedicine diagnosis system according to an embodiment of the present disclosure;

FIG. 5 is a flowchart illustrating a personalization-based telemedicine diagnosis method according to an embodiment of the present disclosure; and

FIG. 6 is a view illustrating the configuration of a computer system according to an embodiment.

### DESCRIPTION OF THE PREFERRED EMBODIMENTS

The advantages and features of the present disclosure and methods of achieving them will be apparent from the following exemplary embodiments to be described in more detail with reference to the accompanying drawings. However, it should be noted that the present disclosure is not limited to the following exemplary embodiments, and may be implemented in various forms. Accordingly, the exemplary embodiments are provided only to disclose the present disclosure and to let those skilled in the art know the category of the present disclosure, and the present disclosure is to be defined based only on the claims. The same reference numerals or the same reference designators denote the same elements throughout the specification.

It will be understood that, although the terms “first,” “second,” etc. may be used herein to describe various elements, these elements are not intended to be limited by these terms. These terms are only used to distinguish one element from another element. For example, a first element discussed below could be referred to as a second element without departing from the technical spirit of the present disclosure.

The terms used herein are for the purpose of describing particular embodiments only and are not intended to limit the present disclosure. As used herein, the singular forms are intended to include the plural forms as well, unless the context clearly indicates otherwise. It will be further understood that the terms “comprises,” “comprising,”, “includes” and/or “including,” when used herein, specify the presence of stated features, integers, steps, operations, elements, and/or components, but do not preclude the presence or addition of one or more other features, integers, steps, operations, elements, components, and/or groups thereof.

In the present specification, each of expressions such as “A or B”, “at least one of A and B”, “at least one of A or B”, “A, B, or C”, “at least one of A, B, and C”, and “at least one of A, B, or C” may include any one of the items listed in the expression or all possible combinations thereof.

Unless differently defined, all terms used herein, including technical or scientific terms, have the same meanings as terms generally understood by those skilled in the art to which the present disclosure pertains. Terms identical to those defined in generally used dictionaries should be interpreted as having meanings identical to contextual meanings of the related art, and are not to be interpreted as having ideal or excessively formal meanings unless they are definitively defined in the present specification.

Hereinafter, embodiments of the present disclosure will be described in detail with reference to the accompanying drawings. In the following description of the present disclosure, the same reference numerals are used to designate the same or similar elements throughout the drawings, and repeated descriptions of the same components will be omitted.

The present disclosure relates to a personalization-technology-based telemedicine diagnosis method and apparatus that uses social-interaction-inducing content for early diagnosis of Autism Spectrum Disorder (ASD).

Specifically, the present disclosure plays a social-interaction-inducing content, which is designed to observe the nonverbal response of a child, using an output device, such as a TV, a monitor, or the like that can be easily accessed at home, childcare facilities, etc., transmits video data, recorded by capturing the response of the child to the content using a recording device, such as a webcam, or the like, to a central server, assists a medical expert in diagnosis by suggesting each nonverbal response detail element and a probability result value for ASD diagnosis based on an AI learning model for early diagnosis of ASD, and personalizes the AI model to suit not only the corresponding assessment subject but also the inclination and unique experience of the medical expert based on few-shot diagnostic values acquired whereby an interpreting physician interprets the same clinical video data, thereby providing higher accuracy in early diagnosis. Also, the AI model according to the present disclosure may be supported to be updated based on the result of diagnosis by the medical expert.

Hereinafter, the present specification presents a telemedicine diagnosis system based on a social-interaction-inducing content platform for reducing the reluctance of parents and children to visit hospitals and undergo diagnosis and assisting in early diagnosis of children with ASD.

The system may play predesigned social-interaction-inducing content and record actions and reactions of a child using an output device, such as a monitor, or the like, and a recording device, such as a webcam, or the like, which can be easily equipped at home or childcare facilities, and may then transmit the recorded video data to a central server.

Subsequently, final interpretation and diagnosis for the clinical video data stored in the server are performed by a medical expert based on AI-assisted diagnosis, and the result thereof is transmitted back to the home, whereby a telemedicine diagnosis service through which parents and children can be provided with a first diagnosis result without the need to visit a hospital may be provided.

Also, in order to overcome the limitations of conventional diagnostic assistive AI models that cannot reflect the patterns of individual patients and the unique experience and inclination of a medical expert because a result value for newly input data is calculated based on parameters pretrained with a large amount of data, a real-time personalized solution based on a small number of diagnostic data values of a medical expert is presented.

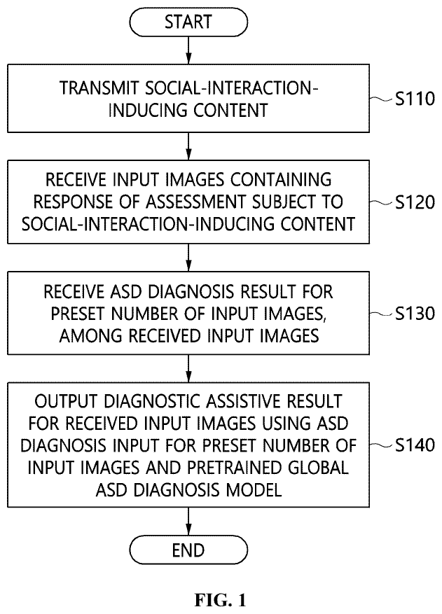

FIG. 1 is a flowchart illustrating a method for assisting in ASD diagnosis according to an embodiment of the present disclosure.

The method for assisting in ASD diagnosis according to an embodiment may be performed by an apparatus for assisting in ASD diagnosis, such as a computing device or a server.

The method for assisting in ASD diagnosis according to an embodiment of the present disclosure includes transmitting social-interaction-inducing content at step S110, receiving input images containing a response of an assessment subject to the social-interaction-inducing content at step S120, receiving an ASD diagnosis result for a preset number of input images, among the received input images, at step S130, and outputting a diagnostic assistive result for the received input images using the ASD diagnosis result for the preset number of input images and a pretrained global ASD diagnosis model at step S140.

Here, receiving the ASD diagnosis result for the preset number of input images, among the received input images, at step S130 may comprise receiving the ASD diagnosis result from a terminal of a medical expert.

Here, outputting the diagnostic assistive result for the received input images at step S140 may include generating a personalized ASD diagnosis mapping model using the pretrained global ASD diagnosis model and the ASD diagnosis result and outputting the diagnostic assistive result using the personalized ASD diagnosis mapping model.

Here, although not illustrated in FIG. 1, in the method, training the global ASD diagnosis model may be performed using the output diagnostic assistive result.

Here, although not illustrated in FIG. 1, the method may further include receiving a result of acceptance of the output diagnostic assistive result.

Here, receiving the result of acceptance of the output diagnostic assistive result may comprise receiving the result of acceptance of the output diagnostic assistive result from the terminal of the medical expert.

Here, although not illustrated in FIG. 1, the method may further include transmitting the diagnostic assistive result to the assessment subject.

Here, transmitting the diagnostic assistive result to the assessment subject may comprise transmitting the diagnostic assistive result to the terminal of the assessment subject.

Here, the ASD diagnosis result may include diagnostic results for eye contact, name call response, a social smile, and pointing.

Here, the global ASD diagnosis model may include respective diagnostic models corresponding to the eye contact, the name call response, the social smile, and the pointing.

Here, outputting the diagnostic assistive result for the received input images at step S140 may comprise generating inference values for the eye contact, the name call response, the social smile, and the pointing based on the respective diagnostic models corresponding to the eye contact, the name call response, the social smile, and the pointing and outputting a personalized diagnostic assistive result using the personalized ASD diagnosis mapping model.

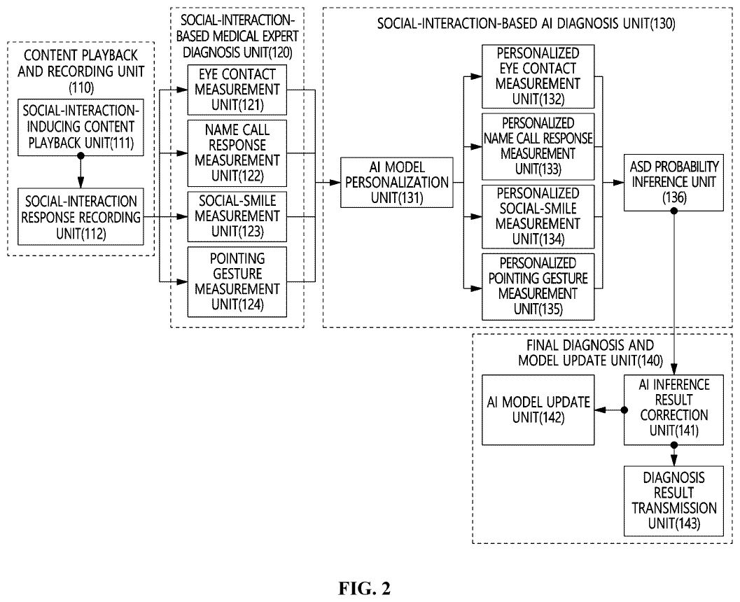

FIG. 2 is a detailed structural diagram of a personalization-based telemedicine diagnosis system for early diagnosis of Autism Spectrum Disorder (ASD).

Referring to FIG. 2, the apparatus according to an embodiment of the present invention includes a content playback and recording unit 110, a social-interaction-based medical expert diagnosis unit 120, a social-interaction-based AI diagnosis unit 130, and a final diagnosis and model update unit 140.

Specifically, the content playback and recording unit 110 includes a social-interaction-inducing content playback unit 111 and a social-interaction response recording unit 112. The social-interaction-inducing content playback unit 111 plays social-interaction-inducing content, which is designed in order to observe nonverbal responses of a child, through an output device, such as a TV, a monitor, or the like, which can be easily accessed at home or childcare facilities.

Here, the content playback and recording unit 110 may be a component corresponding to the terminal of an assessment subject.

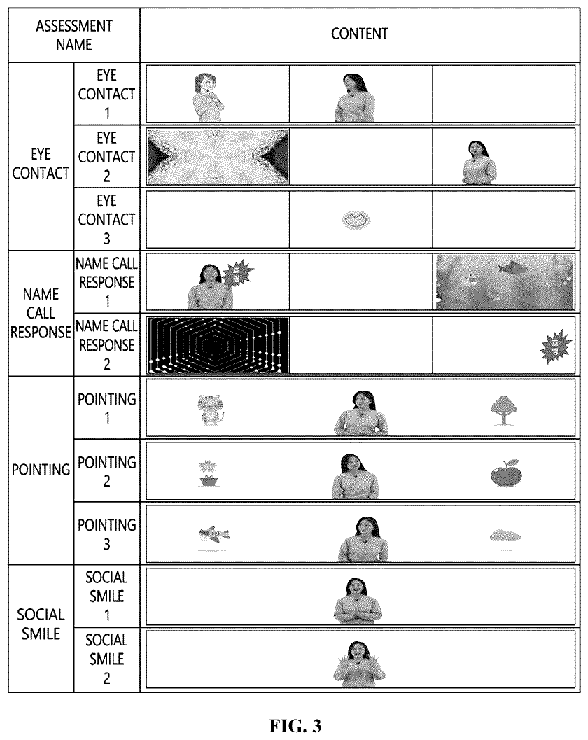

FIG. 3 conceptually illustrates components of social-interaction-inducing content.

Here, the social-interaction-inducing content may be designed to measure the ability to respond to social interaction elements of a child, in particular, the nonverbal elements (eye contact, name call response, a social smile, and pointing) of the child, through consultation with a medical expert, as shown in FIG. 3. Because the ability to socially communicate with others (e.g., between the child and parents or between the child and the medical expert) is determined based on the four detailed elements illustrated in FIG. 3, whether the child positively responds within a given time may be observed through a query-answer format of forced prompting.

Also, each of the detailed elements may be tried several times in order to reduce noise caused by external factors and to increase the reliability of diagnosis. The social-interaction response recording unit 112 may capture the response of the child to the content using a camera recording device, such as a webcam, or the like, and transmit the recorded clinical video data to a central server, and the clinical video data may be captured from a single camera view or simultaneously captured from multiple camera views depending on the environment in which the camera recording system is installed.

The social-interaction-based medical expert diagnosis unit 120 may diagnose the social interaction ability of the child based on the clinical video data recorded by and transmitted from the content playback and recording unit 110. Specifically, the social-interaction-based medical expert diagnosis unit 120 may include four subcomponents, which are an eye contact measurement unit 121, a name call response measurement unit 122, a social-smile measurement unit 123, and a pointing gesture measurement unit 124.

The eye contact measurement unit 121 measures the eye contact ability of a child. Here, eye contact is nonverbal communication in which two people look at each other's eyes, and it plays a role in reading the intention of the other person or sharing one's own intention and interest with the other person.

The name call response measurement unit 122 measures the ability of a child to respond to name calling. Here, the name call response refers to the behavior of the child looking at the person who calls the name of the child, and when the name is called while the child is not looking at the caller, if the child turns his or her head to look at the caller and makes eye contact, it may be determined that the child makes a meaningful name call response.

The social-smile measurement unit 123 measures the facial expression of a child toward others, and may determine whether the child shows an appropriate range of social smiles to an examiner or parents in order to convey an emotional or cognitive state. The pointing gesture measurement unit 124 determines the ability of the child to make a pointing gesture. Here, pointing refers to the behavior of extending a finger in a specific direction or towards a person or an object in order to attract the attention of others, and is one of the most frequently used nonverbal behaviors for communication.

Here, when the medical expert directly performs observation and diagnosis for a large number of test cases stored in the server, which incurs a significant amount of time and cost. For example, in the field of medical image analysis using diagnostic assistive AI, which has recently been actively applied in the medical sector, the diagnostic assistive AI performs the first diagnosis on the medical image to be analyzed, and a medical expert performs correction and the final diagnosis using an inferred result value. As a result, the time required for examination and diagnosis may be efficiently reduced, and it may be expected that the reliability and accuracy of the final diagnosis are improved through the determination based on the AI inference result.

However, in spite of the high potential for the use of diagnostic assistive AI, most of the above-mentioned conventional techniques use a pretrained global AI model to calculate a result value for a new input test case. Accordingly, these techniques fail to reflect not only the characteristics of each patient but also the inclination and unique experience of a medical expert who makes the final diagnosis, and have limitations that reduce susceptibility of the medical expert to the result of assistive diagnosis by AI.

In the present disclosure, before a result of first inference by diagnostic assistive AI is received, a result of manual diagnosis performed by a medical expert on only a small number of test cases, among a large number of recorded test cases in the server, is received as input, and the corresponding data is transferred to the social-interaction-based AI diagnosis unit 130. Here, the number of test cases on which manual diagnosis is performed may be set to an arbitrary value by the medical expert.

The social-interaction-based AI diagnosis unit 130 may include an AI model personalization unit 131, a personalized eye contact measurement unit 132, a personalized name call response measurement unit 133, a personalized social-smile measurement unit 134, and a personalized pointing gesture measurement unit 135.

The AI model personalization unit 131 infers nonverbal response detail elements through global AI models f_{g}^{eye}, f_{g}^{call}, f_{g}^{smile}, and f_{g}^{point} for the same test cases as the small number of test cases, which are manually diagnosed by the medical expert in the social-interaction-based medical expert diagnosis unit 120, and trains a personalized model fₚ that maps the inferred result to diagnosis by the medical expert.

This is for overcoming the above-mentioned limitation in which the global AI model, which is pretrained and stored in the server, fails to reflect the individual characteristics of each patient and the unique experience and diagnostic inclination of a medical expert and for deriving an inference value to which the individual characteristics of the patient and the expert are reflected for the remaining test cases by generating a personalized model through which the result of diagnosis performed by the AI model on a small amount of data is mapped to the result of diagnosis by the expert.

Here, various solvers may be applied in order to generate a personalized model, but parameters in a closed form may be simply estimated using a pseudo inverse method in order to ensure real-time interaction between the AI model and diagnosis by the medical expert. After the personalized mapping model is generated in this way, the personalized eye contact measurement unit 132, the personalized name call response measurement unit 133, the personalized social-smile measurement unit 134, and the personalized pointing gesture measurement unit 135 apply the personalized model to the inference value X for the nonverbal response detail elements derived by the existing global models f_{g}^{eye}, f_{g}^{call}, f_{g}^{smile}, and f_{g}^{point}, thereby calculating a personalized inference value fₚ(X) for each of the nonverbal response detail elements. Additionally, an ASD probability inference unit 136 receives the inference value for each of the nonverbal response detail elements as input and applies a global AI model f_{g}^{ASD} for ASD diagnosis, thereby deriving a probability result value f_{g}^{ASD}(fₚ(X)) for ASD diagnosis.

The final diagnosis and model update unit 140 may include an AI inference result correction unit 141, a diagnosis result transmission unit 143, and an AI model update unit 142.

Specifically, the AI inference result correction unit 141 may receive the inference for each of the nonverbal response detail elements, which is performed by the personalized model, and the probability value for ASD diagnosis, after which the AI inference may be corrected by a medical expert. That is, the medical expert modifies or accepts the final diagnosis result based on the first diagnosis value provided by the personalized AI model. This process may significantly reduce the time taken for the expert to make diagnosis for a large number of test cases and may increase the reliability and accuracy of diagnosis.

The diagnosis result transmission unit 143 transmits the final diagnosis result derived through the above-described method to the server, and the assessment subject checks the diagnosis result by remotely accessing the same server regardless of the place. Accordingly, the assessment subject may be provided with the first screening result in the form of a telemedicine diagnosis service without the need to visit a hospital.

The AI model update unit 142 updates the parameters of the global AI model f_{g}^{ASD} stored in the server based on the diagnostic data of the medical expert, which is derived through the above workflow, thereby improving the performance of the AI model.

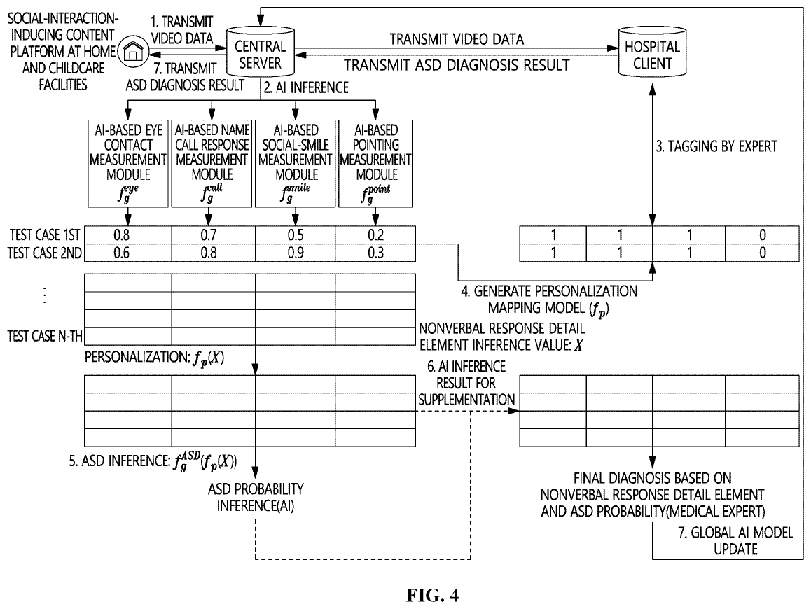

FIG. 4 illustrates a personalization-based telemedicine diagnosis system according to an embodiment of the present disclosure.

Referring to FIG. 4, it can be seen that the personalization-based telemedicine diagnosis system according to an embodiment of the present disclosure includes all of the content playback and recording unit 110, the social-interaction-based medical expert diagnosis unit 120, the social-interaction-based AI diagnosis unit 130, and the final diagnosis and model update unit 140.

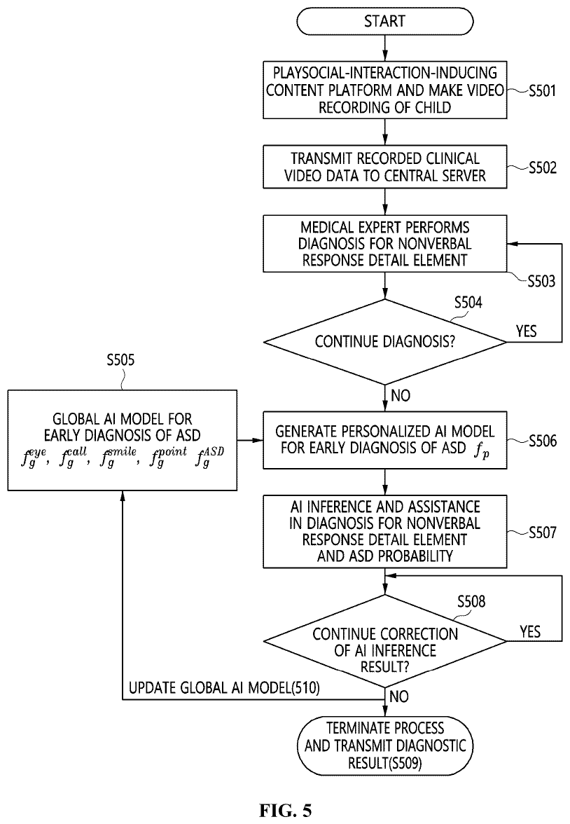

FIG. 5 is a flowchart illustrating a method for personalization-based telemedicine diagnosis according to an embodiment of the present disclosure.

Referring to FIG. 5, first, using output devices and recording devices installed in a home, childcare facilities, and the like, a social-interaction-inducing content is played and a response of a child thereto is recorded at step S501. Subsequently, the recorded clinical video data is transmitted to a central server at step S502, and a medical expert performs manual diagnosis for nonverbal response detail elements (eye contact, name call response, a social smile, and pointing) based on a small number of test cases, among the clinical video data stored in the server, at step S503.

Here, the number of test cases based on which manual diagnosis is performed may be arbitrarily set by the medical expert, and the diagnosis is repeated at step S504 until diagnosis for all of the set number of test cases is completed. Subsequently, a personalized model that maps the value inferred through a global AI model at step S505 to a diagnostic value of the medical expert for the same test cases is generated at step S506, and AI inference values for the nonverbal response detail elements and an ASD diagnosis probability based on the personalized AI model are derived for the remaining test cases at step S507.

Based on the diagnostic assistive data by AI, which is provided through the above-described method, the medical expert modifies or accepts the diagnostic assistive data at step S508, thereby performing final diagnosis. The final diagnosis result is transmitted back to the server at step S509, so that the assessment subject may remotely read the result, and the global AI model is updated based on the result of the final diagnosis by the medical expert at step S510, whereby the performance of the AI model may be continuously improved.

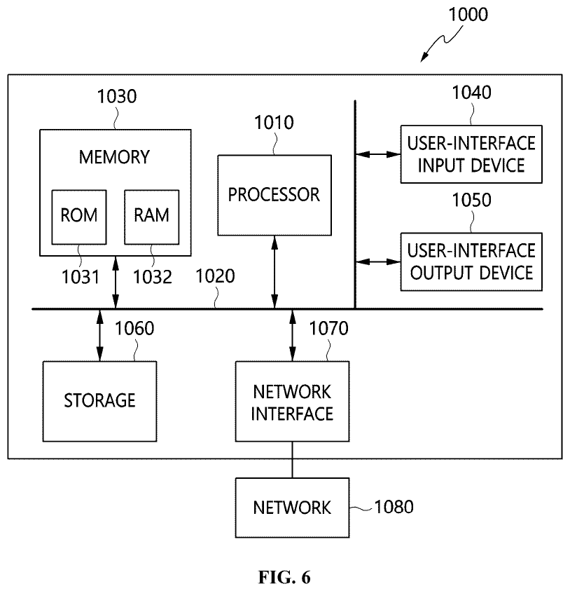

FIG. 6 is a view illustrating the configuration of a computer system according to an embodiment.

The apparatus for assisting in Autism Spectrum Disorder (ASD) diagnosis according to an embodiment may be implemented in a computer system 1000 including a computer-readable recording medium.

The computer system 1000 may include one or more processors 1010, memory 1030, a user-interface input device 1040, a user-interface output device 1050, and storage 1060, which communicate with each other via a bus 1020. Also, the computer system 1000 may further include a network interface 1070 connected with a network 1080. The processor 1010 may be a central processing unit or a semiconductor device for executing a program or processing instructions stored in the memory 1030 or the storage 1060. The memory 1030 and the storage 1060 may be storage media including at least one of a volatile medium, a nonvolatile medium, a detachable medium, a non-detachable medium, a communication medium, or an information delivery medium, or a combination thereof. For example, the memory 1030 may include ROM 1031 or RAM 1032.

The apparatus for assisting in ASD diagnosis according to an embodiment of the present disclosure includes one or more processors 1010 and executive memory 1030 for storing at least one program executed by the one or more processors, and the at least one program includes instructions for performing steps of transmitting social-interaction-inducing content, receiving input images containing a response of an assessment subject to the social-interaction-inducing content, receiving an ASD diagnosis result for a preset number of input images, among the received input images, and outputting a diagnostic assistive result for the received input images using ASD diagnosis input for the preset number of input images and a pretrained global ASD diagnosis model.

Here, outputting the diagnostic assistive result for the received input images may include generating a personalized ASD diagnosis mapping model using the pretrained global ASD diagnosis model and the ASD diagnosis result and outputting the diagnostic assistive result using the personalized ASD diagnosis mapping model.

Here, the program may train the global ASD diagnosis model using the output diagnostic assistive result.

Here, the program may further include an instruction for performing a step of receiving a result of acceptance of the output diagnostic assistive result.

Here, the program may further include an instruction for performing a step of transmitting the diagnostic assistive result to the assessment subject.

Here, the ASD diagnosis result may include diagnostic results for eye contact, name call response, a social smile, and pointing.

Here, the global ASD diagnosis model may include respective diagnostic models corresponding to the eye contact, the name call response, the social smile, and the pointing.

Here, outputting the diagnostic assistive result for the received input images may comprise generating inference values for the eye contact, the name call response, the social smile, and the pointing based on the respective diagnostic models corresponding to the eye contact, the name call response, the social smile, and the pointing and outputting a personalized diagnostic assistive result using the personalized ASD diagnosis mapping model.

According to the present disclosure, supportive results for assisting a medical expert in ASD diagnosis based on an AI neural network may be provided.

Also, the present disclosure may provide supportive results for assisting in ASD diagnosis that reflect the individual characteristics of an assessment subject and a medical expert.

Specific implementations described in the present disclosure are embodiments and are not intended to limit the scope of the present disclosure. For conciseness of the specification, descriptions of conventional electronic components, control systems, software, and other functional aspects thereof may be omitted. Also, lines connecting components or connecting members illustrated in the drawings show functional connections and/or physical or circuit connections, and may be represented as various functional connections, physical connections, or circuit connections that are capable of replacing or being added to an actual device. Also, unless specific terms, such as “essential”, “important”, or the like, are used, the corresponding components may not be absolutely necessary.

Accordingly, the spirit of the present disclosure should not be construed as being limited to the above-described embodiments, and the entire scope of the appended claims and their equivalents should be understood as defining the scope and spirit of the present disclosure.

What is claimed is:

1. A method for assisting in Autism Spectrum Disorder (ASD) diagnosis based on Artificial Intelligence (AI), performed by an apparatus for assisting in ASD diagnosis, comprising:
transmitting social-interaction-inducing content;
receiving input images containing a response of an assessment subject to the social-interaction-inducing content;
receiving an ASD diagnosis result for a preset number of input images, among the received input images; and
outputting a diagnostic assistive result for the received input images using ASD diagnosis result for the preset number of input images and a pretrained global ASD diagnosis model.

2. The method of claim 1, wherein outputting the diagnostic assistive result for the received input images includes:
generating a personalized ASD diagnosis mapping model using the pretrained global ASD diagnosis model and the ASD diagnosis result; and
outputting the diagnostic assistive result using the personalized ASD diagnosis mapping model.

3. The method of claim 1, wherein, in the method, the global ASD diagnosis model is trained using the output diagnostic assistive result.

4. The method of claim 1, further comprising:
receiving a result of acceptance of the output diagnostic assistive result.

5. The method of claim 1, further comprising:
transmitting the diagnostic assistive result to the assessment subject.

6. The method of claim 1, wherein the ASD diagnosis result includes diagnostic results for eye contact, name call response, a social smile, and pointing.

7. The method of claim 2, wherein the global ASD diagnosis model includes respective diagnostic models corresponding to eye contact, name call response, a social smile, and pointing.

8. The method of claim 7, wherein outputting the diagnostic assistive result for the received input images comprises generating inference values for the eye contact, the name call response, the social smile, and the pointing based on the respective diagnostic models corresponding to the eye contact, the name call response, the social smile, and the pointing and outputting a personalized diagnostic assistive result using the personalized ASD diagnosis mapping model.

9. An apparatus for assisting in Autism Spectrum Disorder (ASD) diagnosis based on Artificial Intelligence (AI), comprising:
one or more processors; and
executable memory for storing at least one program executed by the one or more processors,
wherein the at least one program includes instructions for performing:
transmitting social-interaction-inducing content,
receiving input images containing a response of an assessment subject to the social-interaction-inducing content,
receiving an ASD diagnosis result for a preset number of input images, among the received input images, and
outputting a diagnostic assistive result for the received input images using ASD diagnosis input for the preset number of input images and a pretrained global ASD diagnosis model.

10. The apparatus of claim 9, wherein outputting the diagnostic assistive result for the received input images includes:
generating a personalized ASD diagnosis mapping model using the pretrained global ASD diagnosis model and the ASD diagnosis result; and
outputting the diagnostic assistive result using the personalized ASD diagnosis mapping model.

11. The apparatus of claim 9, wherein the program trains the global ASD diagnosis model using the output diagnostic assistive result.

12. The apparatus of claim 9, wherein the program further includes an instruction for performing receiving a result of acceptance of the output diagnostic assistive result.

13. The apparatus of claim 9, wherein the program further includes an instruction for performing transmitting the diagnostic assistive result to the assessment subject.

14. The apparatus of claim 9, wherein the ASD diagnosis result includes diagnostic results for eye contact, name call response, a social smile, and pointing.

15. The apparatus of claim 10, wherein the global ASD diagnosis model includes respective diagnostic models corresponding to eye contact, name call response, a social smile, and pointing.

16. The apparatus of claim 15, wherein outputting the diagnostic assistive result for the received input images comprises generating inference values for the eye contact, the name call response, the social smile, and the pointing based on the respective diagnostic models corresponding to the eye contact, the name call response, the social smile, and the pointing and outputting a personalized diagnostic assistive result using the personalized ASD diagnosis mapping model.
